SEQUENCE LISTING (23 sequences)

SEQ ID NO: 1 - protein, 21 aa, Mus sp.
KEGYKVRLDLNITLEFRTTSK

SEQ ID NO: 2 - protein, 22 aa, Artificial Sequence
  Description of Artificial Sequence Synthetic peptide
RPVRHAQCRVCDGNSTNPRERH

SEQ ID NO: 3 - protein, 21 aa, Artificial Sequence
  Description of Artificial Sequence Synthetic peptide
KNLEISRSTFDLLRNSYGVRK

SEQ ID NO: 4 - protein, 19 aa, Artificial Sequence
  Description of Artificial Sequence Synthetic peptide
TSLRKALLHAPTGSYSDGQ

SEQ ID NO: 5 - protein, 17 aa, Artificial Sequence
  Description of Artificial Sequence Synthetic peptide
KATPMLKMRTSFHGCIK

SEQ ID NO: 6 - protein, 17 aa, Artificial Sequence
  Description of Artificial Sequence Synthetic peptide
DGKWHTVKTEYIKRKAF

SEQ ID NO: 7 - protein, 21 aa, Artificial Sequence
  Description of Artificial Sequence Synthetic peptide
KEGYKVRLDLNITLEFRTTSK

SEQ ID NO: 8 - protein, 22 aa, Artificial Sequence
  Description of Artificial Sequence Synthetic peptide
KQNCLSSRASFRGCVRNLRLSR

SEQ ID NO: 9 - protein, 21 aa, Artificial Sequence
  Description of Artificial Sequence Synthetic peptide
KEGYKVRLDLNITLEFRTTSK

SEQ ID NO: 10 - protein, 21 aa, Artificial Sequence
  Description of Artificial Sequence Synthetic peptide
KEGYKVRLDLNTTLEFRTTSK

SEQ ID NO: 11 - protein, 21 aa, Artificial Sequence
  Description of Artificial Sequence Synthetic peptide
KEGYKVQSDVNITLEFRTSSQ

SEQ ID NO: 12 - protein, 16 aa, Artificial Sequence
  Description of Artificial Sequence Synthetic peptide
KEGYKVRLDLNITLEF

SEQ ID NO: 13 - protein, 12 aa, Artificial Sequence
  Description of Artificial Sequence Synthetic peptide
VRLDLNITLEFR

SEQ ID NO: 14 - protein, 13 aa, Artificial Sequence
  Description of Artificial Sequence Synthetic peptide
LDLNITEFRTTSK

SEQ ID NO: 15 - protein, 21 aa, Artificial Sequence
  Description of Artificial Sequence Synthetic peptide
AEGYAVALDLNITLEFATTSA

SEQ ID NO: 16 - protein, 21 aa, Artificial Sequence
  Description of Artificial Sequence Synthetic peptide
KEGYKVELDLNITLEFETTSK

SEQ ID NO: 17 - protein, 21 aa, Artificial Sequence
  Description of Artificial Sequence Synthetic peptide
KEGYKVELDLNITLEFRTTSK

SEQ ID NO: 18 - protein, 21 aa, Artificial Sequence
  Description of Artificial Sequence Synthetic peptide
KEGYKVRLDLNITLEFETTSK

SEQ ID NO: 19 - protein, 21 aa, Artificial Sequence
  Description of Artificial Sequence Synthetic peptide
KAGYKVRLALNITLAFRTTSK

SEQ ID NO: 20 - protein, 21 aa, Artificial Sequence
  Description of Artificial Sequence Synthetic peptide
KEGYKVRLALNITLEFRTTSK

SEQ ID NO: 21 - protein, 21 aa, Artificial Sequence
  Description of Artificial Sequence Synthetic peptide
KEGYKVRLDLNITLAFRTTSK

SEQ ID NO: 22 - protein, 12 aa, Artificial Sequence
  Description of Artificial Sequence Synthetic peptide
VQSDVNITLEFR

SEQ ID NO: 23 - protein, 5 aa, Artificial Sequence
  Description of Artificial Sequence Synthetic peptide
YIGSR

(19) United States
(12) Patent Application Publication
(10) Pub. No.: US 2010/0285146 A1
(43) Pub. Date: Nov. 11, 2010

(54) PEPTIDES AND METHODS FOR THE TREATMENT OF SYSTEMIC LUPUS ERYTHEMATOSUS

(76) Inventor: Yaakov Naparstek, Jerusalem (IL)

Correspondence Address: EDWARD LANGER;c/o SHIBOLETH YISRAELI ROBERTS ZISMAN & CO., 1 PENN PLAZA-SUITE 2527, NEW YORK, NY, 10119, US

(21) Appl. No.: 12/601,261

(22) PCT Filed: May 22, 2008
(86) PCT No.: PCT/IL08/00698

§ 371 Date: Nov. 22, 2009

Related U.S. Application Data
(60) Provisional application No. 60/939,869, filed on May 24, 2007.

(51) Int. Cl. A61K 35/16; B01J 20/22; B01D 15/08

(52) U.S. Cl. 424/530; 210/198.2

(57) ABSTRACT

A method is disclosed for treating systemic lupus erythematosus in a mammalian subject, comprising administering to said subject an effective dose of at least one laminin peptide, or an analog or a derivative thereof. In one exemplary embodiment, the laminin peptide is selected from the group consisting of R38 (SEQ. ID. NO. 1), and claimed R38 analogs and derivatives thereof including 5200 (SEQ. ID. NO. 10), 5104 (SEQ. ID. NO. 15), 5105 (SEQ. ID. NO. 16), 5106 (SEQ. ID. NO. 17), 5107 (SEQ. ID. NO. 18), 5108 (SEQ. ID. NO. 19), 5109 (SEQ. ID. NO. 20), 5110 (SEQ. ID. NO. 21). The laminin peptides of the present invention may be prepared by known chemical synthetic methods or by biotechnological methods. The invention also provides assays useful for the diagnosis of and following pathological activity course of systemic lupus erythematosus in patients suffering therefrom. In addition, the subject invention concerns a method of treating systemic lupus erythematosus in a subject comprising the extracorporeal removal of lupus antibodies from the subject's plasma and returning the plasma to the subject. In an additional aspect, the invention provides method of reducing anti-R38 antibody levels in a patient's plasma.

### FIELD OF THE INVENTION

This invention relates to the use of laminin peptides and laminin derivatives, including R38 peptide and related analogs for the treatment and detection of systemic lupus erythematosus. This invention also provides methods of treating systemic lupus erythematosus. The invention further provides methods of reducing levels of anti-R38 antibodies in a patient's plasma.

### BACKGROUND OF THE INVENTION

Systemic lupus erythematosus (SLE) is an autoimmune disease involving multiple organs. Through the involvement of the kidneys in the autoimmune inflammatory process, lupus glomerulonephritis is a major cause of morbidity and mortality in this disease (Alarcon-Segovia D. in: Primer on the Rheumatic Diseases. Ed. Schumascher, H. R. Arthritis Foundation, Atlanta, Ga. (1988) pp. 96-100).

Serologically, the disease is characterized by the occurrence of a variety of autoantibodies in the serum, of which the most prominent are the anti-DNA auto antibodies (Naparstek Y., et al., Ann. Rev. Immunol. (1993), 11, 79-104). Although low titers of anti-DNA antibodies may occur in various inflammatory and autoimmune diseases, high levels are found mainly in SLE, and the combination of high anti-DNA antibodies with low complement levels is virtually diagnostic of SLE (Wallace, D. J. et al. in: Dubois' Lupus Erythematosus, Lea and Febiger, Philadelphia, (1993)).

The binding of immunoglobulins to the glomerular basement membrane (GBM) has been shown by the staining of kidneys derived from lupus patients or lupus stains of mice (Wallace, D. J. et al., supra). It has also been shown that anti-DNA antibodies eluted from the kidneys of a lupus patient as well as from MRL/1pr/1pr mice cross-react with sulfated glycosaminoglycans whereas the serum anti-DNA antibodies do not show this cross-reactivity (Naparstek, Y., et al., Arthritis Rheum. (1990), 33, 1554-1559). These results have suggested that extracellular matrix (ECM) plays a role in the pathogenesis of lupus as the target for the nephritogenic autoantibodies.

Termmat R. M. et al. disclose the cross-reaction of components of the ECM, like laminin and heparin with murine monoclonal anti-DNA antibodies. (J. Autoimmun. (1990), 3, 531-545). European Patent Application 670,495 discloses the presence of anti-ECM antibodies in the urine of patients with active lupus. Furthermore, EP 670,495 discloses the cross-reaction of these antibodies with a 200 kDa laminin component of the ECM, and an assay for SLE based on the detection of these anti-ECM/laminin antibodies in urine.

R38 is a peptide sequence isolated from the C-terminal region of the mouse laminin ∝ chain (residues 2890-2910 according to Skubitz et al., J. Cell. Biol. (1991), 115, 1137-1148, or residues 2851-2871 according to Sasaki, M. et al., J. Biol. Chem. (1998), 263, 16, 536-16, 544). It is located at the junction of the globular domains of the fourth and fifth loops (peptide GD-2 in Skubitz et al., supra, and is comprised of the following amino-acid sequence:

| | |
|---|---|
| KEGYKVRLDLNITLEFRTTSK | (SEQ. ID. NO. 1) |

Current SLE therapy is limited to corticosteroids which suppress the over-reactive immune system. This therapy is not specific and its inevitable side effects may themselves be fatal. Furthermore, immunosuppressive therapy is complicated and its initiation is based on a combination of clinical symptoms, blood serological test and kidney biopsy. There is, therefore, a need for a more specific therapy for SLE that will not have the side effects of immunosuppresive agents, as well as a more specific and less invasive assay for the evaluation of disease activity. Indeed, a recent review (The Lancet (1995), 310, 1257-1261) stated that blood tests, though useful in confirming diagnosis of SLE, are “less useful in monitoring disease activity.”

None of the above-mentioned references disclose the treatment of SLE by the administration of the R38 peptide or analogs thereof. Moreover, none of the above-mentioned references disclose the use of R38 peptide in a diagnostic test for SLE or in monitoring SLE disease activity. The contents of all these patents and all literature references referred to above are hereby incorporated by reference in their entirety.

### SUMMARY OF THE INVENTION

It is therefore an object of the present invention to provide a method for treating systemic lupus erythematosus comprising the administration of laminin peptides.

Another object of the present invention is to provide a method of treating SLE comprising the extracorporeal removal of anti-R38 (and derivatives thereof) antibodies from a subject's plasma and returning the plasma to the subject.

Yet another object of the present invention is to disclose R38′ and other novel analogs and derivatives of the R38 peptide, the administration of which comprises a method for treating SLE.

A further object of the present invention is to provide a diagnostic test for SLE by using the R38 peptide, R38′ peptide and other structurally related analogs and derivatives thereof.

The invention also relates to pharmaceutical compositions comprising the R38 peptide, R38′ peptide and other novel analogs and derivatives of the R38 peptide, or pharmaceutically acceptable salts thereof for use in the treatment of SLE.

As used herein, the term, “R38 peptide,” is used to include the R38 peptide itself, analogs, derivatives and fragments thereof that retain the activity of the complete peptide. The term, “analogs,” is intended to include variants on the peptide brought about by, for example, homologous substitution of individual or several amino acid residues. The term, “derivative,” is used to include minor chemical changes that may be made to R38 itself or analogs thereof that maintain the biological activity of R38 and similarly, the term, “fragments,” is used to include shortened molecules of R38.

### BRIEF DESCRIPTION OF THE FIGURES

FIG. 1 shows the direct binding of C72 murine anti-DNA antibody to laminin peptides;

FIG. 2 shows the inhibition by R38, 5200, DNA, DNase and heparin of the binding of C72 to the R38 analog 5200 (sometimes referred to herein as R38);

FIGS. 3 and 4 show the binding of the human lupus monoclonal anti-DNA antibodies (DIL6 and B3) to laminin peptides and derivatives thereof;

FIGS. 5, 6 and 7 show the correlation between lupus activity score and urinary anti-R38 level in three lupus patients;

FIG. 8 shows the effect of 5200 (R38′) treatment on the prolongation of survival of lupus mice;

FIG. 9 shows the effect of R38 (also referred to herein as 5100) treatment on the prolongation of survival of lupus mice;

FIG. 10 shows the inhibition of C72 binding to R38 (5100) by DNA and by R38 analogs;

FIG. 11 is a graph illustrating the relationship of serum antibody concentration to OD measurement;

FIG. 12 is a graph illustrating the change in serum anti-R38 antibody levels pre- and post-treatment in a patient;

FIGS. 13-22 are graphs illustrating individual patient data on serum anti-R38 antibodies pre- and post-treatment using the methods and device of the present invention;

FIG. 23 is a schematic diagram of an embodiment of the invention showing the use of the immunoadsorption column of the invention, with the arrow “A” pointing to the column;

FIG. 24 is a photograph of an adsorption column of the present invention; and

FIG. 25 is a graph showing C72 Anti-R38 antibody binding on the adsorption column of the invention.

### DESCRIPTION OF THE PREFERRED EMBODIMENTS

As used herein in the specification and claims, including as used in the examples and unless otherwise expressly specified, all numbers may be read as if prefaced by the word “about”, even if the term does not expressly appear. Also, any numerical range recited herein is intended to include all sub-ranges subsumed therein.

This application explicitly incorporates by reference all material in the appendix attached hereto, which is a document entitled “Investigator's Brochure” dated Jul. 25, 2006 Ver.001. Accordingly, the present invention relates to the use of laminin peptides for the treatment of systemic lupus erythematosus (SLE).

The present invention is based on the observation that the R38 peptide, which is a peptide derived from the C-terminal region of the mouse laminin cc chain, is recognized by pathogenic lupus antibodies and may therefore possess therapeutic potential in the treatment of SLE by competing in the binding to the lupus antibodies.

Furthermore, the present invention relates to the use of a mixture of at least two or more different peptides derived from laminin for the treatment of systemic lupus erythematosus. In a preferred embodiment, at least one of the peptides is R38 or an analog thererof.

The present invention also provides methods of treating a subject having SLE by the extracorporeal removal of lupus antibodies (anti-R38 and derivatives thereof) from a subject's plasma and returning the plasma to the subject. In one embodiment, the antibodies are removed by column chromatography wherein at least one type of peptide is adsorbed to the column. In a further embodiment, the column is adsorbed with two or more types of peptides. In another embodiment, the peptide is selected from the group consisting of SEQ. ID. NO. 1-22. In a further embodiment, the peptide has SEQ. ID. NO. 1. In yet another embodiment, the peptide has SEQ. ID. NO. 10. In one embodiment, the column is a NHS-activated Sepharose™ High Performance Column.

The invention also relates to a method of monitoring disease activity of patients suffering from SLE, comprising detecting the ability of the antibodies in the urine to bind to the R38 component of the laminin. This binding can have a direct correlation to disease activity evaluated by a combination of various laboratory parameters.

An increase in the amount of antibodies binding to R38 may indicate an approaching active phase of SLE and a declining level of antibodies may indicate an approaching remission. Therefore, this method provides an assay for detecting changes in the level of laminin-specific antibodies and may enable the initiation of therapy prior to the onset of an active phase of the disease.

This method also provides an easy assay that can be used by the patients themselves as it is performed using urine and does not require venipuncture. It may be used as a diagnostic assay, a routine assay for evaluation of SLE disease activity, for early identification of disease exacerbation and for early therapeutic intervention in lupus nephritis.

The R38 peptide or analogs, fragment or derivatives thereof may be used in such an assay using the methods described in EP 670,495. Thus, the R38 peptide may be bound to a solid phase and incubated with the urine from a patient. If the patient is suspected of suffering from SLE, suffering from SLE or is approaching an active phase of the disease, the level of R38-binding antibodies in the urine will increase.

Detection of R38-binding antibodies may be undertaken by any method known by one skilled in the art. Examples of such methods of detection include ELISA and variations thereon, chemiluminescent techniques, etc. The actual method of detection is not crucial to the success of the assay. The level of R38-binding antibodies observed may then be compared to values observed in a control group. The control group may consist of healthy volunteers, or the patient may act as an internal control i.e., the observed value is compared to an earlier value from the same patient. In this manner, a profile of the patient's disease state may be complied and uses as an indicator of further active phases or remission states of the disease.

Pharmaceutically acceptable salts of the R38 peptide include both salts of the carboxy groups and the acid addition salts of the amino groups of the peptide molecule. Salts of the carboxy groups may be formed by methods known in the art and include inorganic salts such as sodium, calcium, ammonium, ferric or zinc salts and the like and salts with organic bases such as those formed with amines such as triethanolamine, arginine or lysine, piperidine, procaine and the like. Acid addition salts include salts with mineral acids such as hydrochloric acid and sulphuric acid and salts of organic acids such as acetic acid and oxalic acid.

The pharmaceutical composition may contain laminin peptides such as the R38 peptide as unique peptides or in polymerized or conjugated forms attached to macromolecular carriers or polymers. The composition may optionally contain pharmaceutically acceptable excipients. In an alternative embodiment, the composition may contain the R38 peptide alone.

The route of administration may include oral, intra-venous, intra-peritoneal, intra-muscular, subcutaneous, intra-articular, intra-nasal, intra-thecal, intra-dermal, trans-dermal or by inhalation.

An effective dose of the R38 peptide or derivatives thereof for use in treating SLE may be from about 1 μg/kg to 100 mg/kg body weight, per single administration, which may be easily determined by one skilled in the art. The dosage may depend upon the age, sex, health and weight of the recipient, kind of concurrent therapy, if any, and frequency of treatment.

In an additional embodiment, the present invention provides a method of reducing the levels of anti-R38 antibodies in a patient's plasma, the method comprising the steps of 1) removing the patient's plasma and passing the plasma through an affinity absorption column comprising a peptide having an amino acid sequence as set forth in SEQ. ID. No. 1, and 2) returning the plasma to the patient's body. Levels of anti-R38 antibodies in the patient's plasma are reduced by over about 60% of pretreatment levels, in some cases over about 80% of pretreatment levels, in other cases over about 90% of pretreatment levels, and in some cases are reduced by over about 95% or even 99% of pretreatment levels of anti-R38 antibodies in the patient's plasma. In the methods of the present invention, all of the plasma passed on the column is returned to the patient, and no plasma replacement is needed.

Advantages of these embodiments of present invention include:
the first lupus-specific treatment is provided—targeting the specific antigen to which the lupus autoantibodies bind.
the methods are highly efficient—removing only the lupus-causing antigen from the plasma, allowing the patient to retain all other vital plasma components.
reduced side effects—as compared to all other existing treatments being used, i.e. immunosuppressive drugs, non-specific plasmapheresis and whole plasma exchange.
operation of the adsorption column is simple and easy.
the treatment is cost-effective—due to the efficiency and effectiveness of the treatment, overall medical costs for severely ill patients are reduced.

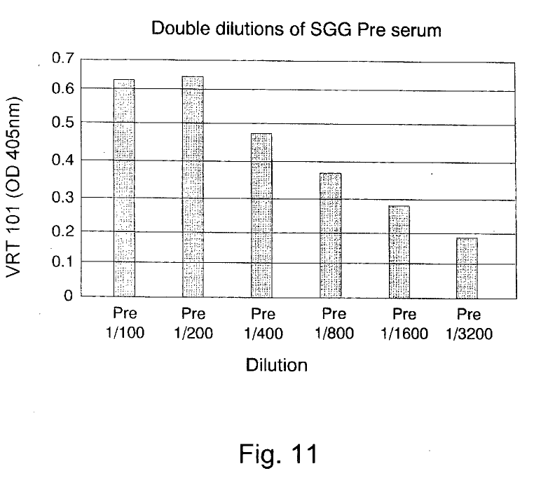

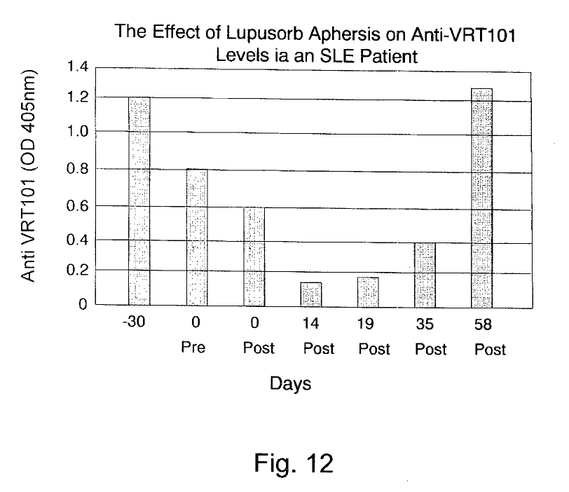

As described in example 13 and shown in FIG. 12, a patient's pretreatment plasma levels of anti-R38 (VRT101) antibodies were about 0.8, and immediately post-treatment were about 0.6, as measured by OD (optical density at 405 nm). At about two-weeks post-treatment anti-R38 antibodies were even further reduced as reflected by an OD measurement of about 0.2. An approximate decrease of over about 70% in the OD measurement (from 0.6 to 0.2) corresponds to over a 97% reduction in plasma antibody concentration, using the information provided in FIG. 11 (relationship of OD measurement to serum concentration of VRT101).

For a period of about one to five weeks, the levels of anti-R38 antibodies remained reduced below immediate post-treatment levels, and slowly returned to pretreatment levels over a period of 7 to 8 weeks. Accordingly, in yet a further embodiment, the present invention provides a method of reducing levels of anti-R38 antibodies in the plasma of a patient by excorporeal treatment of the patient's plasma with an affinity absorption column comprising a peptide having an amino acid sequence as set forth in SEQ. ID. No. 1, wherein the levels of antibody are reduced below immediate post-treatment levels for a period of about one to about five weeks, and increasing gradually thereafter to pretreatment levels. The percent change in antibody levels from immediate post-treatment levels to two weeks post treatment is greater than 80%, in some cases greater than 90%, in some cases greater than 95% or 97%. Further, antibody levels do not return to pretreatment levels for at least 3 weeks, 4 weeks, or in some cases 5 or 6 weeks.

In an additional aspect, the present invention provides an antigen-specific immunoadsorption column for use in the extracorporeal treatment methods of the present invention. The column comprises 1) a ligand peptide having an amino acid sequence as set forth in SEQ. ID. NO. 1, or variants or derivatives thereof, and 2) a column containing the matrix to which the ligand peptide is covalently bound. In one embodiment, the immunoadsorption column of the present invention is the Lupusorb™ column.

The Lupusorb™ column comprises a plastic casing containing a matrix of Sepharose™ beads to which the ligand peptide, R38 is covalently bound forming a DV2 adsorber type single use medical device, responsible for the reversible binding of anti-R38 antibodies in human plasma during a plasmapheresis procedure.

The Lupusorb™ Column is designed to be used during standard plasmapheresis procedure. The upper and lower outlets of the Lupusorb™ column are standard connectors to fit in the corresponding inlet or outlets lines of the plasmapheresis machine. In a routine plasmapheresis procedure consists of removal of blood, separation of blood cells from plasma, and return of these blood cells to the body's circulation, diluted with fresh plasma or a substitute. It is used to remove antibodies from the bloodstream, thereby preventing them from attacking their targets.

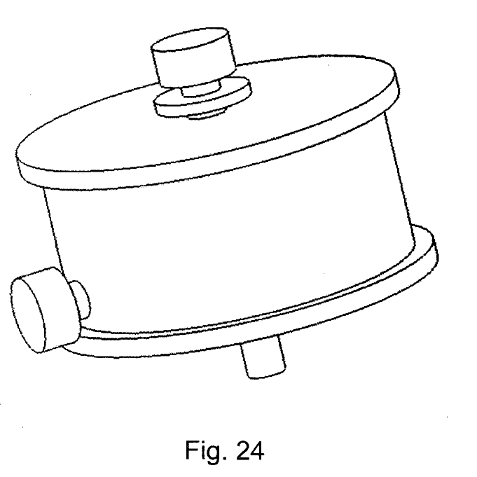

The present invention provides the first plasmapheresis procedure specific to Lupus autoantibodies. In this procedure, the Lupus patient's plasma is passed through a VRT101 (R38)-immunoadsorption column (FIG. 24). In the methods of the present invention, a patient's plasma can be returned to the patient, without the need for dilution with fresh plamsa or a plasma substitute.

The pathogenic lupus autoantibodies are removed by binding to the column, and the rest of the patient's plasma is then re-transfused, without any loss of vital plasma components. R38 was conjugated to the CNBr activated sepharose to form the Lupusorb™ column, consisting of a plastic casing containing a matrix of sepharose beads to which the VRT101 ligand is covalently bound forming a 52 ml adsorber type, single use medical device, responsible for the reversible binding of a respective pathogen in human plasma during routing plasmapheresis procedure.

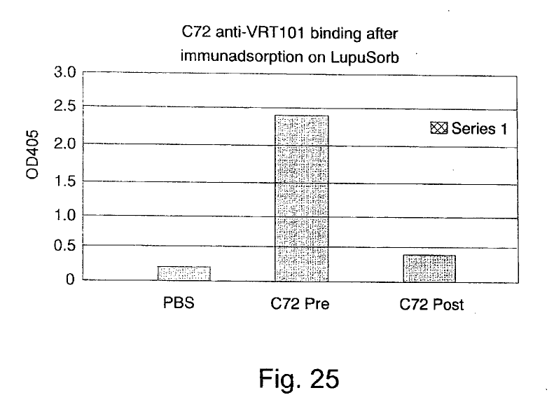

The casing is made from a defined polycarbonate and contains PTFE and PET membranes. All casing materials were required to:
fulfill the requirements of the USP for class VI plastics)
fulfill the requirements of EN ISO 10993 for the intended use,
are suitable for sterilization by ethylene oxide,
are resistant to the pH values used during regeneration,
   The column casing consists of three separately manufactured parts (FIG. 25): a top piece, a centerpiece and a bottom piece. All three parts are ultimately combined by a technique without adhesives.

### Casing Size Specifications:

| | |
|---|---|
| Outer Diameter (Top & Bottom piece) | 65 mm |
| Inner Diameter (Centerpiece) | 55 mm |
| Height | 30 mm |

The top piece is fitted with a central female luer connector, closed with a corresponding luer cap, a circular area with ventilation holes) which are closed off by a tear-off label on the outside and a hydrophobic membrane as a sterility barrier on the inside, and a single membrane layer (PET, 11 μm) covering the complete interior diameter of the top piece.

The central female luer connector guarantees a secure and distinguishable fitting for the tubing system with a male luer lock used for plasma transport (inflow line) to the adsorber during apheresis. The risk of misconnections, plasma loss and contamination is reduced by this form of standard connector. It is closed by a cap until use.

To avoid a pressure build-up and subsequent air pockets in the matrix during treatment and regeneration, the casing is self-ventilating through a circular arrangement of holes in the lid. This circle is closed by a label after filling the adsorbers to prevent evaporation of the saline solution and subsequent deterioration of the sepharose suspension. To prevent contamination of the adsorber contents during treatment and also during storage, the ventilation holes are closed off on the inside by a hydrophobic sterility barrier.

The membrane layer across the whole inner diameter of the adsorber is capable of retaining sepharose from entering into the inflow connector of the adsorber during storage, transportation and handling. In addition, it supports an even distribution of the incoming plasma across the surface of the sepharose.

The centerpiece makes up for the actual volume of the adsorber and is a single polycarbonate cylinder with a female luer connector on the side for filling with sepharose during manufacturing. The connector is closed with a corresponding cap. The outflow tubing will not fit this connector, so that there is no danger of sepharose entry into the return line and on to the patient.

The bottom piece is fitted with a central male luer connector and layers of PET membranes with a pore size of 11 μm.

The standard central male luer connector is specified to fit only with the corresponding part on the outflow line of the tubing set to prevent mismatching of inflow and outflow lines. It is also closed by a cap until use.

The membrane's pore size of 11 μm is sufficiently small to retain the beads of the selected agarose gel matrix, such as Sepharose™, which has a size distribution from 45 to 165 μm. In addition it is mandatory, as a second and independent safety system, to use a sterile particle filter with a pore size of 5 μm between the outflow connector and the outflow tubing.

The chromatography matrix is specified to be a bead-forming agarose-based gel filtration matrix suitable for the coupling of peptides or antibodies in sufficient quantities with a high physical and chemical stability, low adsorption and flow rates in accordance with medically safe and technically achievable plasma flow rates.

In one embodiment, a commercial Sepharose™ 4FF was selected for use in Lupusorb adsorbers. The 4FF Sepharose™ is specified to be suitable for sterilization, have a low bioburden, withstand the pH ranges of human plasma and the regeneration solutions, withstand the chemicals used for coupling of the ligands, provide adequate binding sites for coupling, show only minimal adsorption, provide flow rates adequate for human plasma separation, have retainable bead sizes, and to have a long shelf life.

The 4FF Sepharose™ is specified for use as a base matrix for affinity chromatography following activation, coupling of ligands and blocking. Sepharose™ 4FF comprises spherical beads containing cross-linked agarose (4%) with bead sizes specified to be more than 95% in a range from 45-165 μm, with a mean of 90 μm. The flow rates obtainable with this sepharose concur with the flow rates technically achievable and medically safe for plasmapheresis, such as flow rates anywhere between 15-25 ml/min. The beads maintain their characteristics in a pH range from 2-12 and up to a pressure of 2.0 bar.

The 4FF Sepharose™ shows a high degree of chemical stability over a wide range of substances, including organic solvents and aqueous solutions used during the manufacture and use of the adsorbers. Also, there is virtually no leakage of degradation products (carbohydrates from the agarose) and no non-specific binding because of a lack of charged groups on the sepharose.

Due to the high degree of crosslinking, the 4FF Sepharose™ can be easily sterilized by autoclaving, starting from a very low bioburden as part of the release specification. In addition, the sepharose is supplied in 20% ethanol to further reduce microbial contamination. Under these conditions the Sepharose™ has a shelf life of 5 years.

### EXAMPLES

### The Peptides

Peptides R26, R28, R30, R31, R35, R37, and R38 (also referred hereinafter as “5100” and “TV 5100”) derived from the C-terminal of mouse laminin ∝ chain, and the R18 peptide derived from the N-terminal of mouse laminin ∝ chain were tested. The peptides are 17-22-mer synthetic peptides, and were prepared by the F-moc technique (Carpino, L. A. & Han, G. Y. (1972), J. Org. Chem., 37, 3404). These peptides could also be produced by methods well known to one skilled in the art of biotechnology. For example, using a nucleic acid selected from the group including DNA, RNA, cDNA, genomic DNA, synthetic DNA, mRNA, total RNA, hnRNA, synthetic RNA, the desired peptides may be produced in live cell cultures and harvested. The sequences of the peptides are presented in the Table 1.

TABLE 1
Laminin Derived Peptides

| | RESIDUES | |
|---|---|---|
| PEPTIDES | (*) | SEQUENCE |
| R18 | 42-63 | RPVRHAQCRVCDGNSTNPRERH |
| | | (SEQ. ID. NO. 2) |
| R26 | 2443-2463 | KNLEISRSTFDLLRNSYGVRK |
| | | (SEQ. ID. NO. 3) |
| R35 | 2547-2565 | TSLRKALLHAPTGSYSDGQ |
| | | (SEQ. ID. NO. 4) |
| R37 | 2615-2631 | KATPMLKMRTSFHGCIK |
| | | (SEQ. ID. NO. 5) |
| R28 | 2779-2795 | DGKWHTVKTEYIKRKAF |
| | | (SEQ. ID. NO. 6) |
| R38 | 2890-2910 | KEGYKVRLDLNITLEFRTTSK |
| | | (SEQ. ID. NO. 7) |
| R30 | 3011-3032 | KQNCLSSRASFRGCVRNLRLSR |
| | | (SEQ. ID. NO. 8) |

(*) Residue designations per Skubitz, supra.

Other laminin peptides used for comparative purposes in the Examples include AS31 (comprising the residues YIGSR), AC15 and F9 (other laminin peptides) and R27 a peptide from the 4^{th} loop of the globular region of the laminin ∝ chain.

Additional peptides which are fragments of, or analogs closely derived from R38 have been constructed and are presented in Table 2 hereinbelow. Peptides 5200 and 5101-5111 disclosed in Table 2 were prepared in the same manner as the peptides of Table 1 hereinabove. The Table 2 peptides comprise R38 (5100), human R38 (5300), fragments of R38, a fragment 5111 derived from 5300 or analogs of R38 wherein one or more point substitutions were made according to techniques which are well known to one skilled in the art. These peptides were constructed to investigate, among other things, the effect on anti-DNA antibody binding activity caused by changing the net charge of the R38 peptide.

TABLE 2
Synthetic Peptides Analogous To Mouse R38 Peptide

| Peptide | | | Net |
|---|---|---|---|
| # | AMINO ACID SEQUENCE | DESCRIPTION | Charge |
| 5100 | KEGYKVRLDLNITLEFRTTSK | Mouse R38 | +2 |
| | (SEQ ID NO. 9) | | |
| 5200 | ICEGYKVRLDLNITLEFRTTSK | Mouse R38 analog | +2 |
| | (SEQ ID NO. 10) | | |
| 5300 | KEGYKVQSDLNITLEFRTSSQ | Human R38 | 0 |
| | (SEQ ID NO. 11) | | |
| 5101 | KEGYKVRLDLNITLEF | Res. 1-16 of 5100 | 0 |
| | (SEQ ID NO. 12) | | |
| 5102 | VRLDLNITLEFR | Res. 6-17 of 5100 | 0 |
| | (SEQ ID NO. 13) | | |
| 5103 | LDLNITEFRTTSK | Res. 8-21 of 5100 | 0 |
| | (SEQ ID NO. 14) | | |
| 5104 | AEGYAVALDLNITLEFATTSA | Ala subst. of 5100 at an | −3 |
| | (SEQ ID NO. 15) | positive a.a. | |
| 5105 | KEGYKVELDLNITLEFETTSK | charge subst. to neg. at 5100 | −2 |
| | (SEQ ID NO. 16) | a.a. 7 and 17 | |
| 5106 | KEGYKVELDLNITLEFRTTSK | charge subst. to neg. at 5100 | 0 |
| | (SEQ ID NO. 17) | a.a. 7 | |
| 5107 | ICEGYKVRLDLNITLEFETTSK | charge subst. to neg. at 5100 | 0 |
| | (SEQ ID NO. 18) | a.a. 17 | |
| 5108 | KAGYKVRLALNITLAFRTTSK | Ala subst. of 5100 at all | +5 |
| | (SEQ ID NO. 19) | negative a.a. | |
| 5109 | KEGYKVRLALNITLEFRTTSK | Ala subst. of 5100 a.a. 9 | +3 |
| | (SEQ ID NO. 20) | | |
| 5110 | KEGYKVRLDLNITLAFRTTSK | Ala subst. of 5100 a.a. 15 | +3 |
| | (SEQ ID NO. 21) | | |
| 5111 | VQSDVNITLEFR | Res. 6-17 of 5300 | −1 |
| | (SEQ ID NO. 22) | | |

a.a. = amino acid

### Monoclonal Antibodies

The C72 murine anti-DNA antibody has been derived from (NZBxNZW) F1 lupus mice by the hybridoma technique as described in Eilat D. et al J. Immunol. (1991) 147 361-368. The monoclonal anti-DNA antibodies DIL6 and B3 were derived from lupus patients by hybridoma techniques as described in Ehrenstein M. R. et al J. Clin Invest. (1994) 93 1787-1799 and Ehrenstein M. R. et al Kidney Inter. (1995) 48 705-711.

It should be understood that the following description contemplates use of antibodies specific to the laminins and to the peptides disclosed herein. Methods for producing peptides specific to the laminin peptides and to R38 and its analogs and derivatives are well known to one skilled in the art. In this regard, specific reference may be had to the text “Antibodies, A Laboratory Manual,” Ed Harlow and David Lane, Cold Spring Harbor Publishing, 1988, the contents of which are incorporated herein by reference. This reference discloses methods which may be used for obtaining monospecific antibodies, i.e., monoclonal antibodies and polyclonal antibodies directed against laminin peptides.

### Anti-Peptide (Direct Binding) ELISA

Wells were coated with 10 μg/ml of the peptides, blocked with 1% BSA (bovine serum albumin) in PBS (pH 7.4), reacted with appropriately diluted plasma or monoclonal antibodies, incubated with anti-human or anti-mouse immunoglobulin enzyme conjugated to alkaline phosphatase and detected by addition of substrate (Sigma 100 Phosphatase Substrate Tablets) and color development using an Organon Teknika Microwell System spectrometer at wavelength of 405 nm.

### Competitive Inhibition Assays

In competitive inhibition assays, the antibodies were incubated with various concentrations of the inhibitor (for example: peptide, DNA, heparin) or with DNase for 45 minutes at room temperature and the remaining binding was then evaluated by ELISA as described heretofore. % inhibition was computed as:

$$\frac{\begin{matrix}O . D . binding without inhibitor - \\ O . D . binding with inhibitor\end{matrix}}{O . D . binding without inhibitor} \times 100 = \% inhibition$$

### Example 1

### Binding of Laminin Peptides to SLE Antibodies

### A: Murine SLE Antibodies Bind to C Terminal Peptides of Laminin ∝ Chain

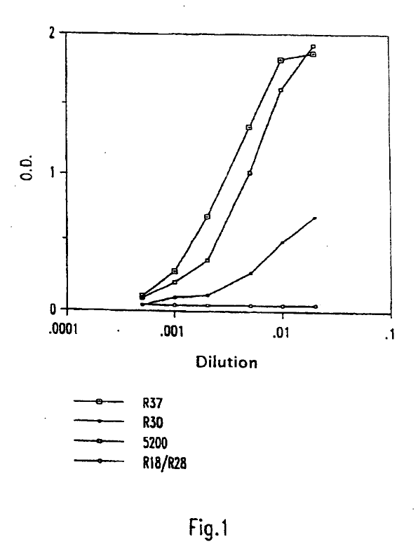

The interaction of the C72 murine anti-DNA antibody with laminin peptides was analyzed by ELISA as described above. The C72 conditioned medium was diluted in PBS in various dilutions. The results are summarized in FIG. 1 which shows the binding of C72 murine anti-DNA antibody to the 5200, R37, and R30 peptides, but not to R28 or R18 peptides of the laminin ∝ chain. Control murine antibody, the anti-HEL Hy5 did not bind to the 5200 peptide (data not shown).

### B: Inhibition of the Binding of C72 to 5200 is Inhibited by DNA and Heparin

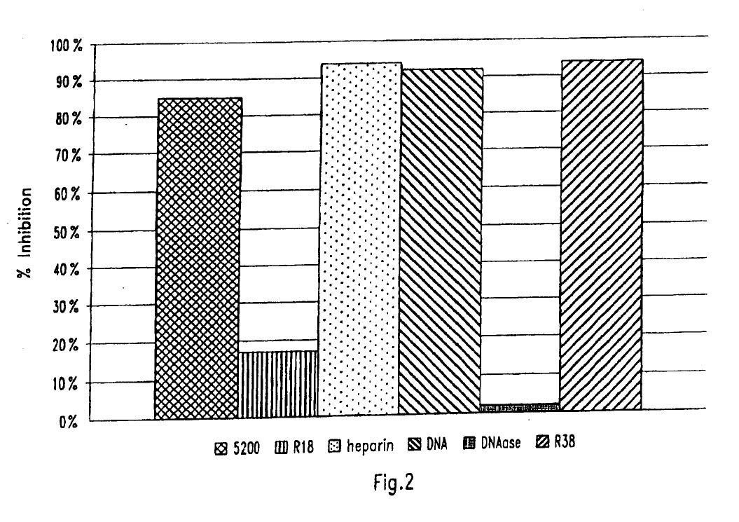

The binding of C72 to 5200 was tested before and after incubation with 5200, R38, R18, Heparin, DNA and DNase. The results are summarized in FIG. 2 which shows the inhibition of the binding of C72 to 5200 by the R38 or 5200 peptides of the present invention, by DNA and by heparin, but not by a control peptide or treatment with DNase. The percent inhibition is the percent reduction of the O.D. after incubation with the inhibiting agent.

### Example 2

### Polyclonal Murine Antibodies Bind to the 5200 Peptide

Analysis of the interaction of MRL/1pr/1pr urine antibodies with the 5200 peptide by a direct binding ELISA revealed specific binding. Thus, pooled urine from at least 5 mice (either MRL/1pr/1pr or control mice, e.g. BALB/c) was added to wells coated with R38′ (5200), R18 or DNA as described above and bound 5200 assayed by ELISA.

### Binding of Murine Urinary Immunoglobulins to 5200

Each group is comprised of pooled urine.

| | ANTIGEN | | |
|---|---|---|---|
| MICE | DNA | 5200 | R18 |
| BALB/c | U.D. | U.D. | U.D. |
| MRL/1pr/1pr | U.D. | 0.26(*) | U.D. |

U.D.—Undetected
(*)O.D. at 405 nm.

### Example 3

### Human Monoclonal Lupus Antibodies Bind the 5200 Peptide

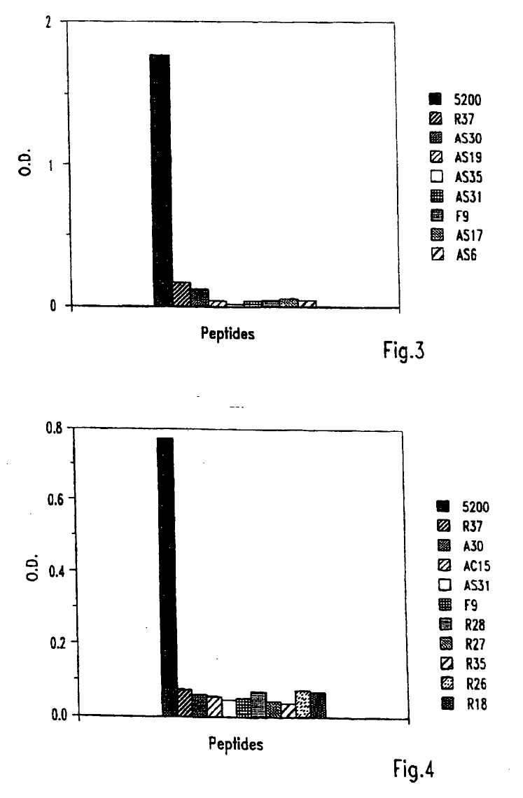

The human monoclonal anti-DNA antibodies DIL 6 and B3 were derived from lupus patients by the hybridoma technique. As shown in FIGS. 3 and 4 these antibodies were found to bind to the 5200 peptide but not to other laminin peptides tested. In FIGS. 3 and 4, the peptides are referred to as denoted above or as follows; AS30 is R27, AS19 is R35, AS35 is R26, AS17 is R28 and AS6 is R18.

### Example 4

### Effect of R38 (5100) & R38′ on the Clinical Course of Murine SLE

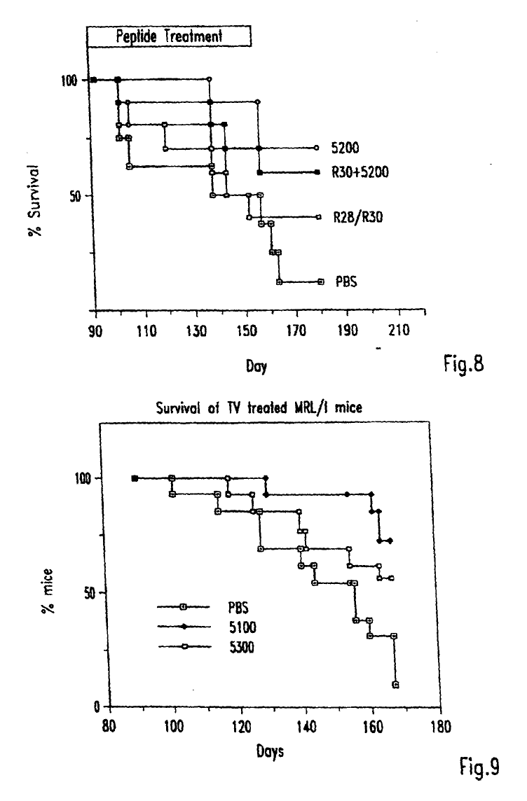

To test whether R38 peptides can affect the course of SLE we have tested their effect on MRL/1pr/1pr mice disease. 60 μg of 5200 (R38′ alone or in peptide combinations, 30 μg of each) in 0.1 ml PBS, was injected i.p. to 6 week old female MRL/1pr/1pr mice once a week for 16 weeks and the mice were evaluated for survival (FIG. 8), and for renal histology.

50 μg of 5100 (R38) or 5300 (human R38) in 0.1 ml PBS, was injected i.p. to 6 week old MRL/1pr/1pr mice three times a week and the mice were evaluated for survival (FIG. 9), and for renal histology. Control mice received 0.1 ml phosphate buffer solution. Each test and control group contained 12-15 mice.

The survival of MRL/1pr/1pr mice treated with 5100, 5200 or 5300 was compared to that of PBS treated mice. As shown in FIGS. 8 and 9, the survival of mice treated with 5100 or 5200 was significantly higher than that of control mice. In FIGS. 8 and 9 the time in days shown on the x axis relates to the age of the mice. Two mice in each group were sacrificed after 5 months and their kidneys evaluated by light microscopy. The kidneys from the control mice showed severe diffuse proliferative glomerulonephritis with crescents and sclerosis whereas the 5100 or 5200 treated mice showed mild proliferative changes with no crescents and no sclerosis.

### Example 5

### Analysis of the Correlation Between Anti-R38Antibodies and Disease Activity

Urine from lupus patients with and without renal disease in active and inactive state were collected repeatedly and tested for presence of anti-R38 antibodies by ELISA. Activity of the disease was evaluated also by accepted clinical and serological parameters (Lockshin M. D. et al. Am. J. Med (1984) 77 893-898) and their correlation with anti-R38 levels was compared. 103 urine samples of 37 SLE patients were tested for anti-R38 activity by ELISA as described above. 23 of the samples were from patients without renal disease and 80 samples from patients with renal disease. A further 12 samples from patients with renal disease not related to SLE were also included.

The following results were obtained:

| | SLE | | |
|---|---|---|---|
| | Present | Present | Absent |
| | Renal Disease | | |
| | Absent | Present | Present |
| No. Samples | 23 | 80 | 12 |
| Urine anti-R38 O.D. | 0.035 ± 0.003 | 0.229 ± 0.03* | 0.07 ± 0.01 |
| (Mean + S.E.) | | | |

*p < 0.001

Positivity of the samples in those patients with renal disease usually correlated with active disease according to an activity score that includes 19 clinical and laboratory parameters (Lockshin M. D. et al. supra). These parameters included assessment of the presence/absence/condition of the following clinical criteria: alopecia, rash, fever, serositis, athralgia/arthritis, mucosal ulcers, neurological events, malaise, fundi changes, nodes, spleen and the following blood tests including ESR (erythrocyte sedimentation rate), anti-DNA antibodies, complement (U/ml), creatinine, haemoglobin (g/dl), PLT platelets (/mm²) or urinalysis. The assessments of these parameters is measured as described in Lockshin supra. The overall percentage given reflects only the assessed parameters.

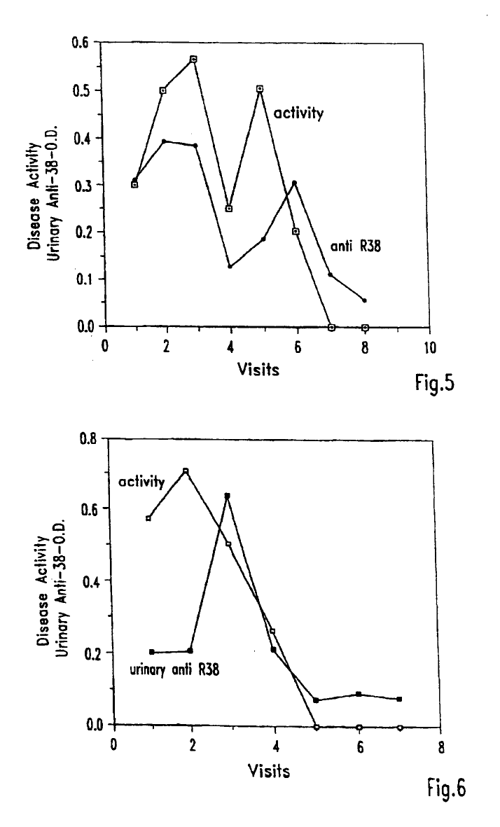

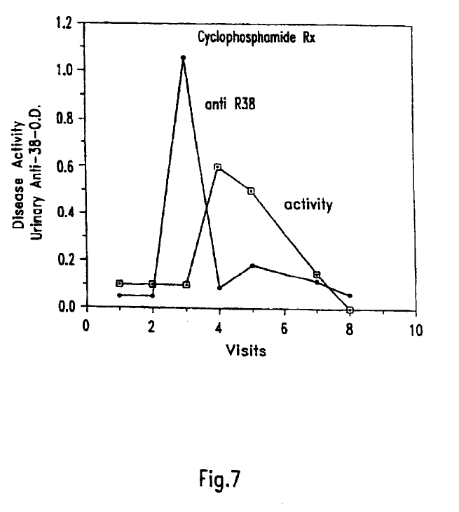

In some patients urine samples were tested in more than one occasion and a good correlation between the clinical activity and the level of anti-R38 binding were observed. Three representative examples from three different lupus patients are shown in FIGS. 5, 6 and 7 where the x-axis shows the No. of the hospital visit and the y-axis, the observed binding (OD at 405 nm) or percentage of the activity score described above. As can be seen from these Figures, the assay using the R38 peptide provides a reliable method of monitoring disease activity.

### Example 6

### Analysis of the Correlation Between Anti-5200 (R38′) Antibodies and Disease Activity

In an additional experiment, 178 urine samples from lupus patients, 24 with and 22 without renal disease in active and inactive state were collected and tested for presence of anti-5200 antibodies by ELISA as described above. The following results were obtained:

Renal Disease

| | Absent | Present |
|---|---|---|
| No. Samples | 46 | 132 |
| Urine anti-5200 O.D. | 0.05 ± 0.005 | 0.335 ± 0.035* |
| (Mean + S.E.) | | |

*p < 0.001

### Example 7

### Analysis of the Correlation Between Anti-5100 (R38) Antibodies and Disease Activity

45 urine samples from 21 lupus patients, some with and some without renal disease in active and inactive state were collected and tested for presence of anti-5100 antibodies by direct ELISA as described above.

The following results were obtained:

Renal Disease

| | Absent | Present |
|---|---|---|
| No. Samples | 6 | 39 |
| Urine anti-5100 O.D. | 0.058 ± 0.006 | 0.376 ± 0.05* |
| (Mean + S.E.) | | |

*p < 0.03

### Example 8

### Analysis of the Correlation Between Anti-5200 (R38′) Antibodies and Disease Activity

52 urine samples from 21 lupus patients, with and without renal disease in active and inactive state were collected and tested for presence of anti-5200 antibodies by ELISA as described above.

The following results were obtained:

Renal Disease

| | Absent | Present |
|---|---|---|
| No. Samples | 6 | 46 |
| Urine anti-5200 O.D. | 0.052 ± 0.03 | 0.431 ± 0.09 |
| (Mean + S.E.) | | |

### Example 9

### Analysis of the Correlation Between Anti-5108, 5101, 5109 and 5110—Antibodies and Disease Activity

24 urine samples from some of the lupus patients of Examples 7 and 8, 2 with and 22 without renal disease in active and inactive state were collected and tested for binding to 5108 peptides by ELISA as described above.

The following results were obtained:

Renal Disease

| | Absent | Present |
|---|---|---|
| No. Samples | 2 | 22 |
| Urine anti-5108 O.D. | 0.064 ± 0.05 | 0.672 ± 0.1 |
| (Mean + S.E.) | | |

Similar results were observed for binding of peptides 5101, 5109 and 5110.

### Example 10

### Direct Binding of C72 and B3 to R38 and Analog Peptides

The peptides of the present invention were tested for their ability to bind directly with C72 murine anti-DNA antibodies and B3 human anti-DNA antibodies according to the method described hereinabove. The results of the direct binding study are reported in Table 3:

TABLE 3
Direct Binding Of C72 And B3 To R38 and Analog Peptides

| Peptide # | C72 Binding′ | B3 Binding |
|---|---|---|
| 5100 | 2.57 | 0.9 |
| 5200 | 1.8 | 0.25 |
| 5300 | 0.03 | 0.03 |
| 5101 | 1.11 | 0.1 |
| 5102 | 0.1 | 0.02 |
| 5103 | 0.03 | 0.02 |
| 5104 | 0.06 | 0.02 |
| 5105 | 0.07 | 0.02 |
| 5106 | 1.9 | 0.16 |
| 5107 | 0.05 | 0.01 |
| 5108 | 2.75 | 1.93 |
| 5109 | 2.72 | 1.94 |
| 5110 | 2.8 | 1.83 |
| 5111 | 0.01 | 0.01 |
| R18 | 0.01 | NT* |
| R28 | 0.01 | NT |
| R30 | 0.75 | NT |
| R37 | 1.8 | NT |

*NT—Not Tested
+ - O.D. in a direct binding ELISA test after one (1) hour.
‡ - O.D. in a direct binding ELISA test after two (2) hours.

### Example 11

### Competitive Inhibition of C72 Binding to R38 with Analog Peptides

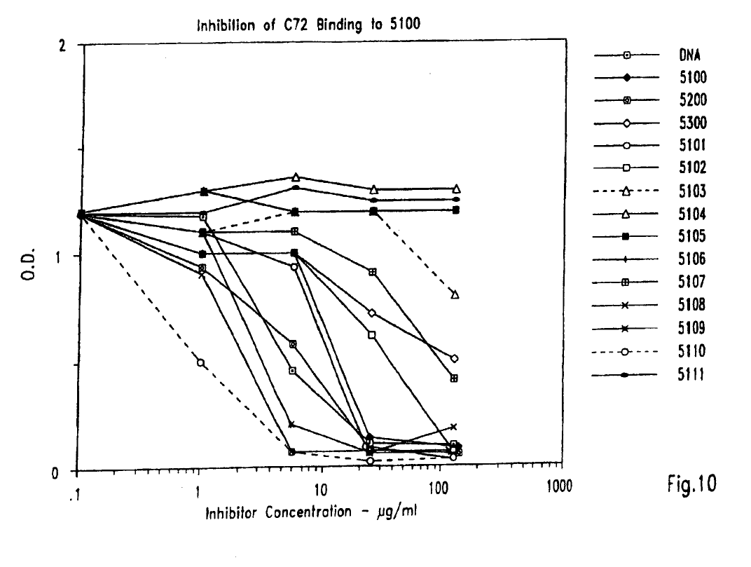

A competitive inhibition study compared how each of the peptides competes with R38 (5100) for binding to the C72 anti-DNA antibody. Conducted according to the methods described hereinabove, the results of the study are disclosed in Table 4 below and are further elucidated by reference to FIG. 10.

TABLE 4
Inhibition of C72 Binding to R38

| | 50% Inhibition of C72 binding |
|---|---|
| Peptide # | to mouse R38 (5100) in ug/ml* |
| 5100 | 10 |
| 5200 | 10 |
| 5300 | 85 |
| 5101 | 5 |
| 5102 | 30 |
| 5103 | NI** |
| 5104 | NI |
| 5105 | NI |
| 5106 | 2.5 |
| 5107 | 85 |
| 5108 | 2 |
| 5109 | 0.7 |
| 5110 | 0.7 |
| 5111 | NI |

*Concentration of competitive inhibitor which resulted in 50% inhibition of the binding of C72 anti-DNA antibody to peptide 5100 (R38) in an ELISA test.
NI**—No Inhibition

### Example 12

### Immunoabsorption of SLE Antibodies on a Column

This example demonstrates the use of a column for extracorporeal removal of anti-R38 (TV-5100) (and derivatives thereof) pathogenic lupus antibodies from a subject's blood.

### Preparation of the Column

The R38 peptide was dissolved in the coupling buffer (0.2M NaHCO₃, 0.5M NaCl, pH 8.3) in a concentration of 1 mg/ml in 5 ml coupling buffer. A 5 ml N-hydroxysuccinimide (NHS)-activated Sepharose™ High Performance Column (Pharmacia 17-0717-01) is used. The isopropanol in the column was washed out from the column with 30 ml of cold (4° C.) 1 mM HCl and 5 ml of the peptide solution was then injected onto the column with a syringe (2.5 ml/minute). The column was sealed and stood for 30 minutes at room temperature. The column was then washed with 30 ml Buffer A (0.5 M ethanolamine, 0.5 M NaCl, pH 8.3) and 30 ml Buffer B (0.5 M acetate, 0.5 M NaCl, pH 4) consecutively three times, and then with neutral pH buffer (0.05 M Na₂HPO₄ and 0.1% NaN₃).

### Affinity Absorption of the Anti-R38 (TV-5100) Antibodies

The column was washed with 15 ml PBS (phosphate buffered saline), followed by 15 ml elution buffer (0.1 M glycine HCl) and then 50 ml PBS. The C72 antibody or the patients' plasma samples were filtered through a 0.45 microm filter. The samples were applied onto the column by a fitted syringe at a rate of 2.5 ml/min. The column was then washed with 5 ml PBS and the flow-through was applied to the column again. The binding of the samples (original and flow-through) to R38 was tested by the anti-R38 ELISA test.

In the instant example, the mouse C72 and the SLE patients' plasma were applied to the R38 column. Their anti-R38 binding was evaluated by ELISA in the original samples and in the flow-through of the column.

As shown in Table 5, affinity absorption on the column removed 99% of the anti-R38 activity of the mouse monoclonal C72 and between 30%-60% of the antibodies in the human SLE patients' plasma.

TABLE 5
Affinity Absorption

| | Binding to R38 (O.D.) | |
|---|---|---|
| Sample | Original sample | After immunoabsorption |
| C72 | 1.96 | 0.02 |
| SLE patient 1 plasma | 1.5 | 0.6 |
| SLE patient 2 plasma | 2.3 | 1.6 |
| Healthy donor's plasma | 0.26 | 0.06 |

Thus, SLE may be treated by affinity absorption of a SLE patient's plasma and returning the plasma to the patient intravenously.

### Example 13

### Phase I/II Clinical Trial with Lupusorb™ Immunoadsorption Columns in Systemic Lupus Erythematosus (SLE or Lupus) Patients

10 SLE patients were treated with a single Lupusorb™ immunoadsoprtion session during routine plasmapheresis procedure. The Lupusorb™ immunoadsorption column is an affinity adsorption column comprising R38 (VRT101) peptides. Patient screening prior to enrollment into the study is between 3 months up to 5 days prior to the day planned for plasmapheresis. Patients are enrolled into the study on the plasmapheresis day and undergo treatment of between 1.5-2.25 hours with the Lupusorb™ column. The patient is then followed up for 8 weeks after the Lupusorb™ column procedure.

The first patient underwent treatment and completed the two month follow-up period. No adverse events were reported and the procedure was well-tolerated. As to preliminary efficacy, FIG. 12 depicts the changes in VRT101 antibody levels of the patient before treatment, after treatment and during the follow-up period. As shown in FIG. 12, the level of anti-VRT (R38) antibodies decreased after the Lupusorb™ apheresis and returned to the original levels after more than 5 weeks.

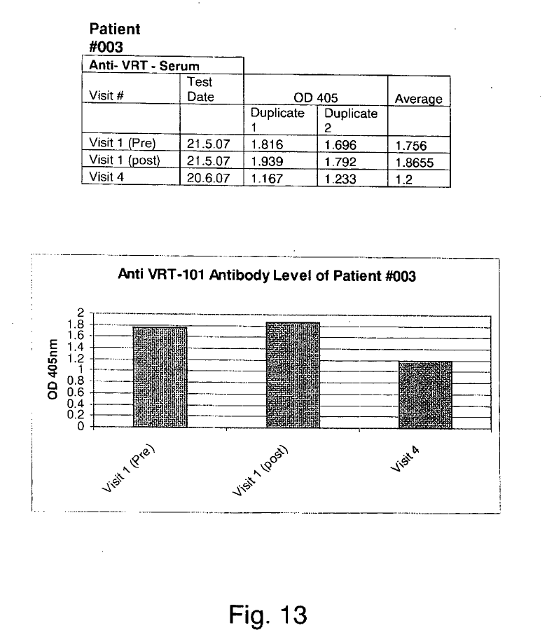

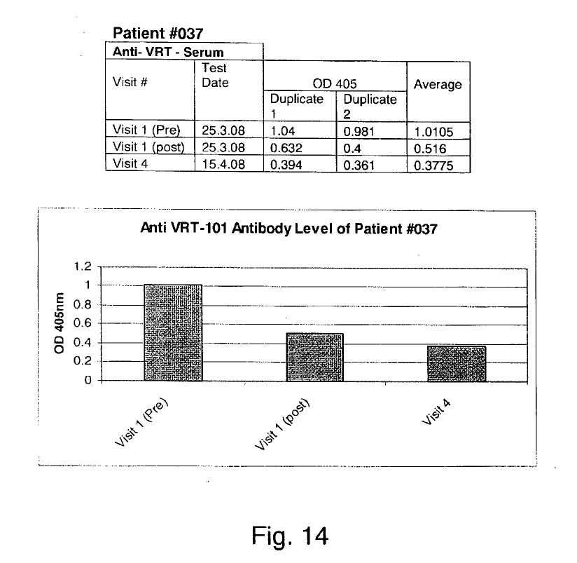

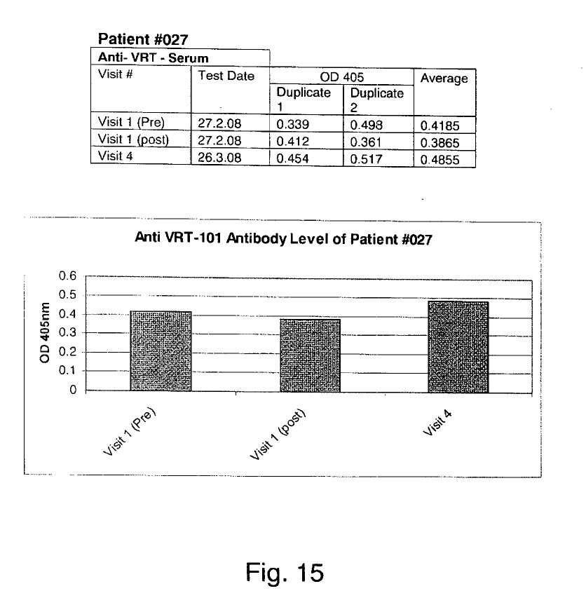

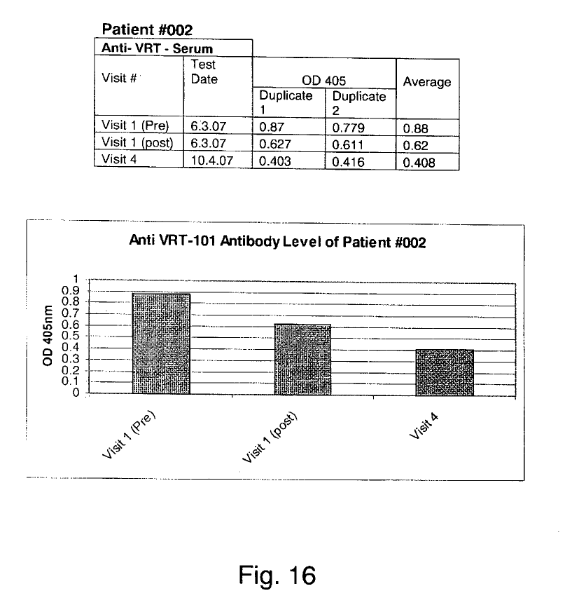

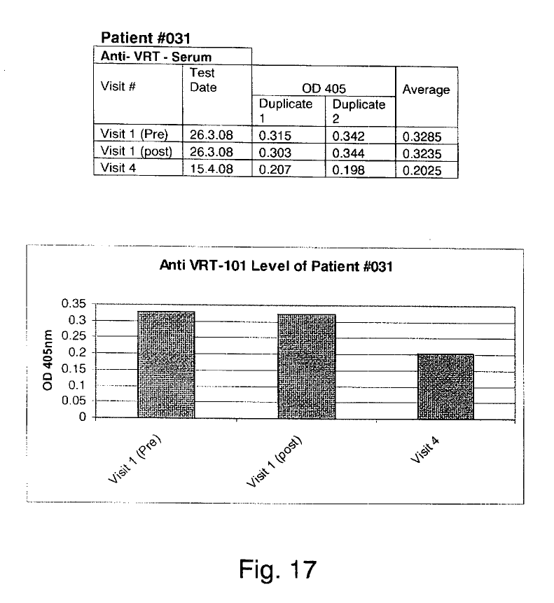

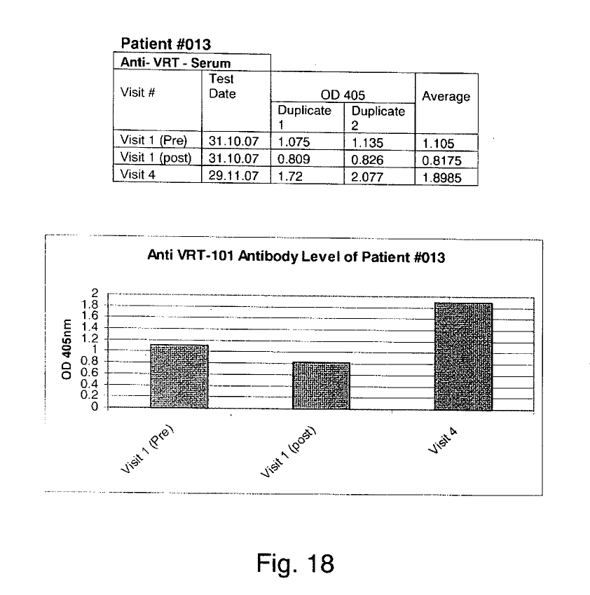

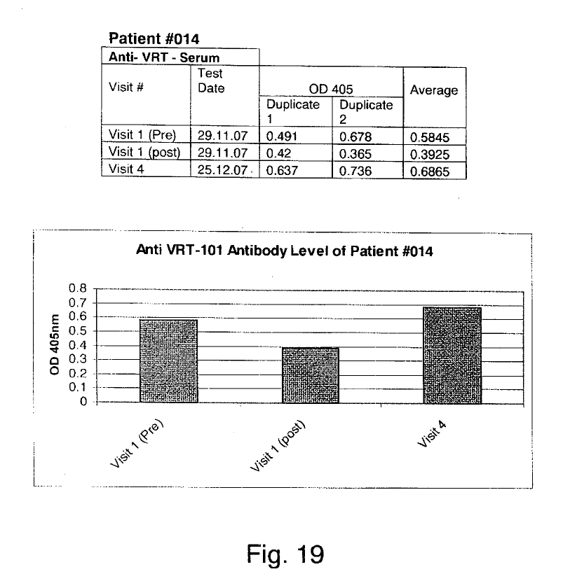

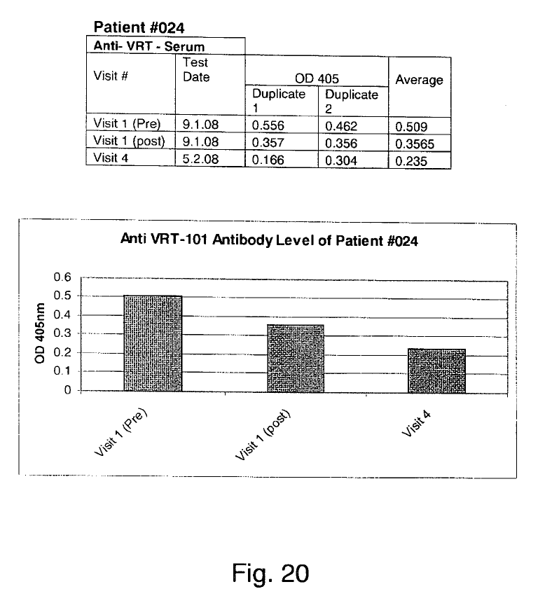

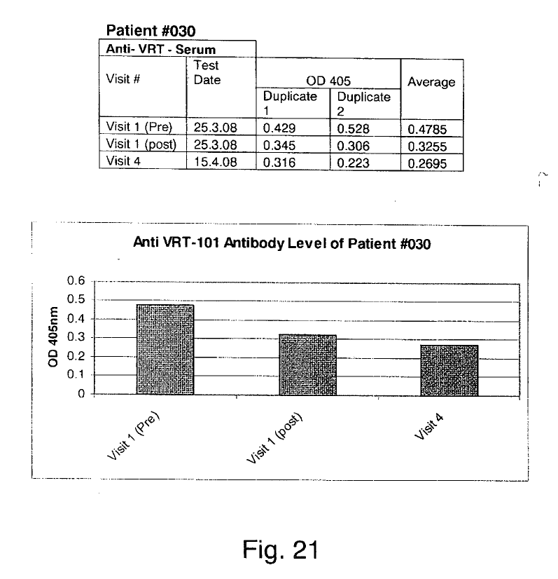

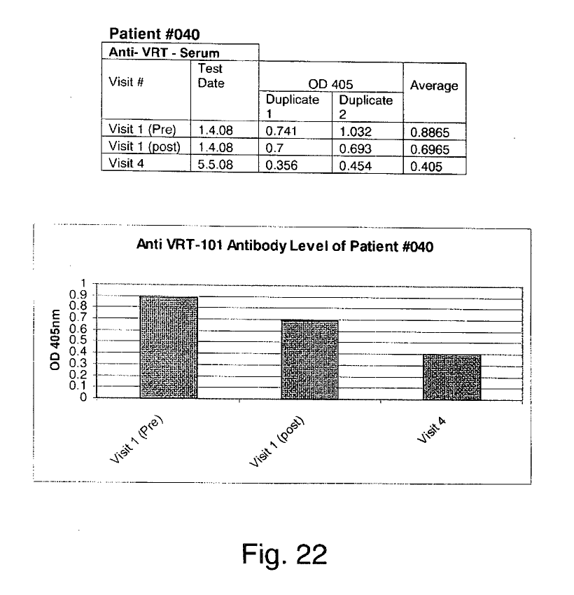

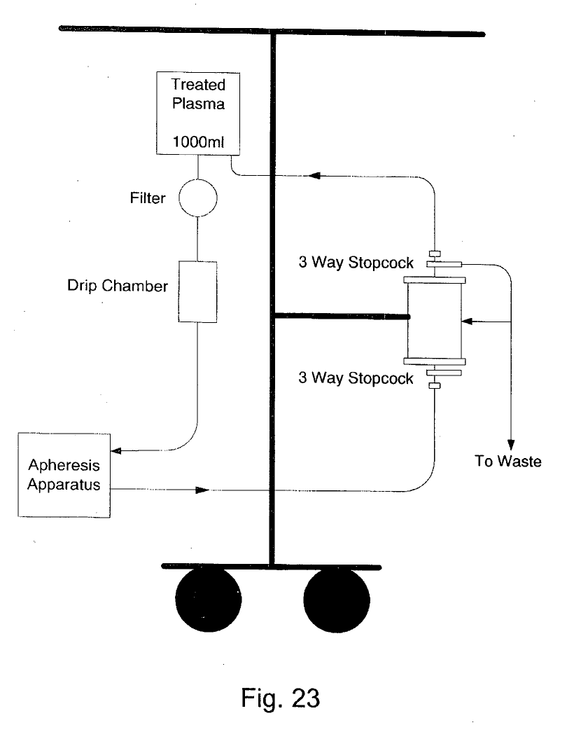

Additional patient information, obtained according to the same protocol, is shown in FIGS. 13-22. Two patients, #013 and 027 show no anti VRT101 level effects of the treatment. The remaining patients all had reduced anti-VRT (R38) antibodies to the normal level shown from follow up visits 2 or 3 until follow up visits 4 or 5, with the exception of patient #003 who showed decreased anti VRT101 level but higher than the norm. There was no significant rebound effect in any of the ten patients.

No serious adverse events related to the treatment of the Lupusorb™ were reported.

### Example 14

Evaluation of anti-VRT (R38) concentration in SLE patient's plasma.

10 ml plasma, of a lupus patent (LM), were loaded on the column. The column was rinsed with PBS (at least 5 column volumes), eluted with 0.05M NaCl in 10 m/M HCL (pH 3.0) and 1 ml fractions were collected. The peak fractions (identified by anti VRT101 binding by ELISA) were 7-9, were tested for IgG content by radial immunodifusion (RID) or by ELISA. The original plasma immunoglobulin amount was 164 mg (based on a concentration 1640 mg/dL) whereas the peak fractions added up to less than 200 μg (concentration of less than 6.9 mg/dL by RID).

These results demonstrate that the specific antibodies represent a very small quantity of the immunoglobulin content, namely, about 0.12% of the total immunoglobulins.

### Example 15

### Lupusorb Column Capacity

In a small scale experiment, the binding capacity of the Lupusorb column by saturation of the LupuSorb column with C72 antibodies was tested as follows: a 5 ml LupuSorb column was loaded with 350 ml of C72 supernatant containing about 12 μg/ml of immunoglobulin. Fractions of the flow-through were collected and tested for VRT101 binding by ELISA.

The first fraction was 60 ml and the following fractions were 30 ml each. As can be seen in FIG. 26, the first fraction shows very little binding to VRT101; The ability of the column to remove the C72 antibody is saturated after 150 ml supernatant (1.8 mg IgG), indicating that the column can bind at least 360 μg immunoglobulin/ml resin.

### Example 16

To test the ability of the LupuSorb column to perform apheresis and define the technical parameters including plasma volume and flow rate we have performed two to three monthly plasmapheresis experiments in three sheep. The sheep were anesthetized and intubated and 3 liters of plasma underwent apheresis. The rate flow was set to 16-20 ml/minute.

There were no problems in the continuous and homogenous flow of the plasma through the column. One sheep died after the second procedure due to anesthesia problems during intubation. The remaining sheep did not show any immediate or late response to the procedure and their hematological and biochemical blood parameters did not show significant changes from the original results.

It should be understood that the foregoing description and examples are merely illustrative and that many modifications and variations may be made thereto by one skilled in art without departing from the scope and spirit of the invention as claimed hereinbelow.

What is claimed:

1. A method of treating a subject having systemic lupus erythematosus comprising the extracorporeal removal of lupus antibodies from the subject's plasma and returning the plasma to the subject, without the need for additional plasma replacement.

2. The method of claim 1, wherein the removal of lupus antibodies is performed by column chromatography on a column adsorbed with at least one type of peptide.

3. The method of claim 2, wherein the peptide is selected from the group consisting of SEQ. ID. NO. 1, SEQ. ID. NO. 2, SEQ. ID. NO. 3, SEQ. ID. NO. 4, SEQ. ID. NO. 5, SEQ. ID. NO. 6, SEQ. ID. NO. 7, SEQ. ID. NO. 8, SEQ. ID. NO. 9, SEQ. ID. NO. 10, SEQ. ID. NO. 11, SEQ. ID. NO. 12, SEQ. ID. NO. 13, SEQ. ID. NO. 14, SEQ. ID. NO. 15, SEQ. ID. NO. 16, SEQ. ID. NO. 17, SEQ. ID. NO. 18, SEQ. ID. NO. 19, SEQ. ID. NO. 20, SEQ. ID. NO. 21, and SEQ. ID. NO. 22.

4. The method of claim 2, wherein the column is adsorbed with two or more types of peptides.

5. The method of claim 3, wherein the peptides are selected from the group consisting of SEQ. ID. NO. 10, SEQ. ID. NO. 19, SEQ. ID. NO. 20, SEQ. ID. NO. 21.

6. The method of claim 3, wherein the peptide has SEQ. ID. NO. 1.

7. The method of claim 3, wherein the peptide has SEQ. ID. NO. 10.

8. The method of claim 3, wherein the column is a N-hydroxysuccinimide (NHS)-activated sepharose column.

9. A method of reducing anti-R38 antibody levels in a patient, the method comprising the steps of
1) removing the patient's plasma and passing the plasma through an affinity absorption column comprising a peptide having an amino acid sequence as set forth in SEQ. ID. No. 1, and
2) returning the plasma to the patient's body,
wherein plasma levels of anti-R38 antibodies in the patient's plasma are reduced by over about 95% of pretreatment levels of anti-R38 antibodies in the patient's plasma.

10. A method of reducing levels of anti-R38 antibodies in the plasma of a patient by extracorporeal treatment of the patient's plasma with an affinity adsorption column comprising a peptide having an amino acid sequence as set forth in SEQ. ID. No. 1, the method comprising the steps of
1) removing the patient's plasma and passing the plasma through an affinity absorption column comprising a peptide having an amino acid sequence as set forth in SEQ. ID. No. 1, and
2) returning the plasma to the patient's body,
wherein the levels of anti-R38 antibodies are reduced below immediate post-treatment levels for a period of about one to about five weeks.

11. An affinity adsorption column comprising a peptide having the amino acid sequence as set forth in SEQ. ID. NO. 1, or variants or derivatives thereof.

12. The adsorption column of claim 11, wherein the column is filled with an agarose-based gel filtration matrix.

13. The adsorption column of claim 12, wherein the agarose-based gel has a particle size of about 45 to 165 μm.

14. The adsorption column of claim 12, wherein the agarose gel is N-hydroxysuccinimide (NHS)-activated.

15. An affinity adsorption column comprising a peptide selected from the group consisting of SEQ. ID. NO. 1, SEQ. ID. NO. 2, SEQ. ID. NO. 3, SEQ. ID. NO. 4, SEQ. ID. NO. 5, SEQ. ID. NO. 6, SEQ. ID. NO. 7, SEQ. ID. NO. 8, SEQ. ID. NO. 9, SEQ. ID. NO. 10, SEQ. ID. NO. 11, SEQ. ID. NO. 12, SEQ. ID. NO. 13, SEQ. ID. NO. 14, SEQ. ID. NO. 15, SEQ. ID. NO. 16, SEQ. ID. NO. 17, SEQ. ID. NO. 18, SEQ. ID. NO. 19, SEQ. ID. NO. 20, SEQ. ID. NO. 21, and SEQ. ID. NO. 22.

16. The adsorption column of claim 15, wherein the column is adsorbed with two or more types of peptides.

17. The adsorption column of claim 15, wherein the peptides are selected from the group consisting of SEQ. ID. NO. 10, SEQ. ID. NO. 19, SEQ. ID. NO. 20, SEQ. ID. NO. 21.
